(19) United States
(12) Patent Application Publication
(10) Pub. No.: US 2017/0183387 A1
(43) Pub. Date: Jun. 29, 2017

(54) METHODS AND COMPOSITIONS FOR SUPPRESSING RETROVIRUSES

(71) Applicant: Irene Hoxie, Millburn, NJ (US)

(72) Inventor: Irene Hoxie, Millburn, NJ (US)

(21) Appl. No.: 15/364,184

(22) Filed: Nov. 29, 2016

Related U.S. Application Data
(60) Provisional application No. 62/260,559, filed on Nov. 29, 2015.

(51) Int. Cl. C07K 14/47; C12N 7/00; C12N 5/0783; C12N 15/86

(52) U.S. Cl. CPC C07K 14/47; C12N 15/86; C12N 7/00; C12N 5/0636; C12N 2710/10342; A61K 38/00

(57) ABSTRACT

The disclosure provides methods and compositions for suppressing retroviruses, including novel methods for treating a retroviral infection in a human in need of such treatment, comprising delivering a functional meiosis arrest female protein 1 (MARF1) to cells containing a retroviral provirus, together with novel expression constructs comprising a coding sequence encoding a functional MARF1 operatively linked to a promoter, vectors comprising such constructs, and packaging cell lines for use in making such vectors.

### CROSS REFERENCE TO RELATED APPLICATION

This application claims priority to U.S. Provisional Application No. 62/260,559, filed Nov. 29, 2015, the contents of which are incorporated herein by reference.

### FIELD

The disclosure relates to the field of gene therapy for treatment and control of retroviral infections, for example HIV infection, and provides methods and compositions therefor.

### BACKGROUND

The Retroviridae are a family of viruses that insert a DNA copy of their genome into the genome of the host cell in order to replicate. A retroviral genome is made up of two single strands of plus-sense RNA. In the retrovirus, the RNA is bound to a capsid protein and encased, along with enzymes necessary for replication, within an envelope made of a retroviral envelope protein in association with glycoproteins and lipids from the membrane of the host cell. The envelope facilitates recognition of the potential host cell and entry into the host cell. Once inside the host cell cytoplasm, the retrovirus releases a reverse transcriptase enzyme to produce DNA from the retroviral RNA genome, which DNA is then incorporated into the host cell genome by a retroviral integrase. The retroviral DNA that is integrated into the host cell genome is called a provirus. The provirus is effectively part of the host cell's genome, translating and transcribing the viral genes along with the cell's own genes, producing the proteins required to assemble new copies of the virus, and transmitting the retroviral DNA to progeny cells.

If a retrovirus integrates into the germline of its host, it can be passed on for generations. These so-called endogenous retroviruses are believed to make up a significant portion of the human genome.

About 45% of the human genome is composed of transposable elements. Retrotransposons are a class of these mobile genetic elements that move and replicate in their host through an RNA intermediate using reverse transcriptase. Long terminal repeat (LTR) retrotransposons in particular have close structural resemblance to retroviruses. They are flanked by long terminal direct repeats that contain all of the necessary transcriptional regulatory elements. The autonomous elements (retrotransposons) contain gag and pol genes, which encode a protease, reverse transcriptase, RNAse H and integrase. The main difference between retroviruses and LTR retrotransposons is that retroviruses include a functional envelope gene, so that they can form transmittable viral particles that can infect other cells.

Retrotranspons may interfere with genome integrity. Mammalian germ cells in particular, have come up with effective tools for retroelement surveillance. In mammals, transposon surveillance is crucial in oocytes during fetal oocyte attrition, a process where about 75% of egg cells are deemed “inadequate” and die. In oocytes, MARF1 functions as a retrotransposon surveillance tool. Mutations of MARF1 cause female infertility.

Human immunodeficiency virus (HIV) is a retrovirus that causes Acquired Immunodeficiency Syndrome (AIDS) in humans. Since 1981, an estimated 25 million people are have died from HIV infections globally. The disease has proven extremely difficult to treat. The viral envelope is made up largely of human glycoproteins and lipids, while the viral envelope protein—the only viral DNA exposed to the immune system—is variable, and the virus may be dormant and virtually undetectable for long periods. This makes the development of effective vaccines challenging.

HIV can be divided into two major types, HIV type 1 (HIV-1) and HIV type 2 (HIV-2). The HIV-1 Group M viruses predominate and are responsible for the AIDS pandemic. HIV-1 Group M can be further subdivided into subtypes based on genetic sequence data. Some of the subtypes are known to be more virulent or are resistant to different medications. HIV-2 viruses are thought to be less virulent and transmissible than HIV-1 Group M viruses, although HIV-2 is also known to cause AIDS. While HIV-1 Group M subtypes C and A make up about 74% of HIV infections globally, most infections in North America and Europe are subtype B.

Glycoproteins in the HIV envelope bind to CD4+ T helper cells, as well as macrophages and dendritic cells. Once bound, the retrovirus enters the cell by membrane fusion and starts transcribing its RNA genome to a DNA genome on its way to the nucleus. The HIV includes integrase, which integrates this DNA into the host genome as a provirus. When activated, the provirus transcribes mRNA, as well as enzymes and structural proteins to make up the virus. The viral mRNA is transported out to the cytoplasm where packaging occurs. The packaged virions then bud out of the host cell, carrying with them lipids and glycoproteins from the host cell membrane, to infect more cells. The HIV provirus may be relatively dormant for long periods, and is able to hide from the immune system by producing undetectable amounts of viral proteins. An unknown signal causes the HIV provirus to “activate,” so that it replicates and releases virus. The infected cells may be killed by the virus directly or via triggering of apoptosis, or they may be destroyed by cytotoxic CD8+ cells that recognize a foreign antigen on the surface of the infected cells. Once the infected cell types, particularly the CD4+ T cells, decline below a critical level, the immune system can no linger provide cell-mediated immunity, and the patient becomes very susceptible to opportunistic infections and cancers

An HIV positive person is diagnosed with AIDS when his or her CD4 cell count falls below 200 cells/μl or CD4 T-lymphocyte percentage of total lymphocytes is less than 14%.

HIV has a unique pseudodiploid genome, as well as low replication fidelity, and a complex viral envelope, making it able to evolve quickly and develop resistance to drugs or vaccines. The viral proliferation can be inhibited by aggressive antiviral therapies such as HAART (Highly Active Antiretroviral Therapy), which is a triple (or more)-drug therapy method combining inhibitors of binding and entry, reverse transcriptase, integration, transcription, assembly, and release. Such combinations therapies are very expensive and debilitating. There is, however, no cure for the disease, so the therapy must be continued for as long as the patient can tolerate the side effects or until the virus evolves to become resistant to the treatment.

New and better alternatives to treatment of retroviral infections are urgently needed.

### SUMMARY

This invention provides a new approach to treatment of retroviral infections, by using MARF1 to silence the retroviral provirus.

The invention provides in some embodiments, methods of treating a retroviral infection in a human in need of such treatment, comprising delivering a functional meiosis arrest female protein 1 (MARF1) to cells containing a retroviral provirus, for example by means of a DNA vector such as an adenoviral (AV) vector. In other embodiments, the invention provides vectors comprising sequences encoding MARF1 and packaging cell lines that express such vectors.

[3] Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### DETAILED DESCRIPTION

The invention this provides, in one embodiment, a method (Method 1) of treating a retroviral infection in a human in need of such treatment, comprising delivering a functional meiosis arrest female protein 1 (MARF1) to cells containing a retroviral provirus. By functional MARF1 is meant a human MARF1 protein or a fragment or variant thereof which retains MARF1 activity in human cells, e.g., which is capable of repressing transposable elements and inhibiting their mobilization.

For example, the invention provides
1.1. Method 1, wherein the MARF1 is delivered by means of a DNA vector comprising an expression cassette having a coding sequence encoding a functional MARF1 operatively linked to a promoter, that which will express MARF1 in said cells containing a retroviral provirus.
1.2. Method 1.1 wherein the DNA vector is a replication-deficient viral vector.
1.3. Method 1.2 wherein the replication-deficient viral vector is an adenoviral (AV) vector.
1.4. Method 1.3 wherein the AV vector is not capable of integration into the genome of said cells containing a retroviral provirus.
1.5. Method 1.2 wherein the replication-deficient viral vector is an adeno-associated viral (AAV) vector.
1.6. Method 1.5 wherein the AAV vector is not capable of integration into the genome of said cells containing a retroviral provirus.
1.7. Any of foregoing Methods 1.1 to 1.6 wherein the DNA vector transiently expresses MARF1 in the cells containing a retroviral provirus but does not integrate into the genome of said cells.
1.8. Any of Methods 1.1, 1.2 or 1.3 wherein the cells containing a retroviral provirus are permanently transformed with a gene constitutively expressing MARF1.
1.9. Any foregoing method wherein the gene expressing a functional MARF1 comprises a cDNA encoding a human MARF1.
1.10. Any foregoing method wherein the MARF1 is a human MARF1.
1.11. Any foregoing method wherein the MARF1 has at least 90% amino acid sequence identity, e.g. at least 95% identity, e.g., at least 99% identity, e.g., as measured by a BLAST algorithm, to a MARF1 selected from GenBank Accession: NP_055462.2, NP_001171927.1 and NP_001171928.1.
1.12. Any foregoing method wherein the MARF1 has at least 90% amino acid sequence identity, e.g. at least 95% identity, e.g., at least 99% identity, e.g., as measured by a BLAST algorithm, to SEQ ID: 1
SEQ ID 1:

| | |
|---|---|
| 1 | mmegngtens csrtrgwlqq dndakpwlwk fsncfsrpeq tlphspqtke ymenkkvave |
| 61 | lkdvpsplha gsklfpavpl pdirslqqpk iqlssvpkvs ccahcpneps tspmrfgggg |
| 121 | ggsggtssli hpgalldsqs trtitcqvgs gfafqsassl qnasarnnla giasdfpsmc |
| 181 | lesnlssckh lpccgklhfq schgnvhklh qfpslqgcts agyfpcsdft sgapghleeh |
| 241 | isqseltphl ctnslhlnvv ppvclkgsly cedclnkpar nsiidaakvw pnipppntqp |
| 301 | aplavplcng cgtkgtgket tlllatslgk aaskfgspev avagqvlenl ppigvfwdie |
| 361 | ncsvpsgrsa tavvqrirek ffkghreaef icvcdisken keviqelnnc qvtvahinat |
| 421 | aknaaddklr qslrrfanth tapatvvlvs tdvnfalels dlrhrhgfhi ilvhknqase |
| 481 | allhhaneli rfeefisdlp prlplkmpqc htllyvynlp ankdgksysn rlrrlsdncg |
| 541 | gkvlsitgcs ailrfinqds aeraqkrmen edvfgnriiv sftpknrelc etkssnaiad |
| 601 | kvkspkklkn pklclikdas eqsssakatp gkgsqansgs atkntrwksl qelcrmeskt |
| 661 | ghrnsehqqg hlrlvvpthg nssaaystpk nsgvaepvyk tsqkkenlsa rsvtsspvek |
| 721 | kdkeetvfqv sypsafsklv asrqvsplla sqswssrsms pnllnraspl afnianssse |
| 781 | adcpdpfang advqvsnidy rlsrkelqql lqeafarhgk vksvelspht dyqlkavvqm |
| 841 | enlqdaigav nslhrykigs kkilvslatg aaskslslls aetmsvlqda pacclplfkf |
| 901 | tdiyekkfgh klnvsdlykl tdtvaireqg ngrlycllps sqarqsplgs sqshdgsstn |
| 961 | cspiifeele yhepvcrqhc snkdfsehef dpdsykipfv ilslktfapq vhsllqtheg |
| 1021 | tvpllsfpdc yiaefgdlev vqenqggvpl ehfitcvpgv niataqngik vvkwihnkpp |
| 1081 | ppntdpwllr skspvgnpql iqfsrevidl lksqpscvip ishfipsyhh hfakqcrvsd |
| 1141 | ygyskliell eavphylqil gmgskrlltl thraqvkrft qdllkllksq askqvivref |
| 1201 | sqayhwcfsk dwdvteygvc elidivseip dtticlsqqd nemvicipkr ertqdeiert |
| 1261 | kqfskdvvdl lrhqphfrmp fnkfipsyhh hfgrqcklay ygftkllelf eaipdtlqvl |
| 1321 | ecgeekiltl teverfkala aqfvkllrsq kdnclmmtdl lteyaktfgy tfrlqdydvs |
| 1381 | sisaltqklc hvvkvadies grqiqlinrk slrsltaqll vllmswegtt hlsveelkrh |
| 1441 | yesthntpin pceygfmtlt ellkslpylv evftndkmee cvkltslylf aknvrsllht |
| 1501 | yhyqqiflhe fsmaytkyvg etlqpktygh ssveellgai pqvvwikghg hkrivvlknd |
| 1561 | mksrlsslsl spanhenqps egerilevpe shtaselklg adgsgpshte qellrltdds |
| 1621 | pvdllcapvp sclpspqlrp dpvilqsadl iqfeerpqep seimilnqee kmeipipgks |
| 1681 | ktltsdssss cisaavpvpp cpssetsesl lskdpvespa kkqpknrvkl aanfslapit |
| 1741 | kl |

1.13. Any foregoing method wherein the MARF1 comprises one or more, or 5 or more, or all of the following conserved sequences from the sequence of SEQ ID NO 1: 352-494, 1487-1562, 1004-1074, 1100-1171, 1260-1330, 1176-1247, 1412-1484, 787-875, 510-582, 1336-1406, 876-937, 795-865.
1.14. Any foregoing method wherein the MARF1 comprises residues 352-1562 of SEQ ID NO 1.
1.15. Any foregoing Method 1.1, et seq. wherein the sequence encoding a functional MARF1 is a sequence which encodes a protein which comprises one or more, or 5 or more, or all of the following conserved sequences from the sequence of SEQ ID NO 1: 352-494, 1487-1562, 1004-1074, 1100-1171, 1260-1330, 1176-1247, 1412-1484, 787-875, 510-582, 1336-1406, 876-937, 795-865; e.g., a protein which comprises residues 352-1562 of SEQ ID 1.
1.16. Any foregoing Method 1.1, et seq. wherein the sequence encoding a functional MARF1 is a sequence which has at least 90% sequence identity, e.g. at least 95% identity, e.g., at least 99% identity, e.g., as measured by a BLAST algorithm, to the coding sequence (CDS) of a nucleotide sequence selected from GenBank Accession: NM_014647.3, NM_001184998.1, and NM_001184999.1.
1.17. Any foregoing Method 1.1 et seq. wherein the promoter is a viral promoter.
1.18. Any foregoing Method 1.1 et. seq. wherein the promoter is selected from cytomegalovirus (CMV) and chicken (3-actin (CBA) promoters.
1.19. Any foregoing Method wherein the retroviral infection is a lentiviral infection.
1.20. Any foregoing Method wherein the retroviral infection is human immunodeficiency virus (HIV), e.g., HIV-1 or HIV-2, e.g., HIV-1, e.g. HIV-1 Group M, one or more of HIV-1 Group M subtypes A, B or C.
1.21. Any foregoing method wherein the cells containing a retroviral provirus are white blood cells.
1.22. Any foregoing method wherein the cells containing a retroviral provirus are CD4+ lymphocytes.
1.23. Any foregoing Method 1.1 et seq. wherein the functional meiosis arrest female protein 1 (MARF1) is delivered to the cells containing a retroviral provirus
   1.23.1. In vivo, e.g., by injection; or
   1.23.2. Ex vivo, e.g., by removing from the patient's body, treating, and returning the cells containing a retroviral provirus.
1.24. Any foregoing a method comprising
   taking blood from the patient, and optionally further isolating cells containing a retroviral provirus from the blood, e.g. CD4+ cells;
   delivering the functional meiosis arrest female protein 1 (MARF1) to the cells containing a retroviral provirus;
   and returning said blood or cells to the patent's circulatory system.
1.25. Any foregoing method wherein the patient receives antiviral medications which will inhibit retroviral expression but will not inhibit AAV vector delivery and expression, e.g., medications selected from one or more of
   1.25.1. Non-nucleoside reverse transcriptase inhibitors (NNRTIs)
   1.25.2. Nucleoside reverse transcriptase inhibitors (NRTIs)
   1.25.3. Protease inhibitors (PIs)
   1.25.4. Fusion inhibitors
   1.25.5. CCR5 antagonists (CCR5s)
   1.25.6. Integrase strand transfer inhibitors (INSTIs)
1.26. Any foregoing method wherein subsequent to treatment with MARF1, the patient receives antiviral medications to inhibit reinfection by the retrovirus, e.g., medications selected from one or more of
   1.26.1. Non-nucleoside reverse transcriptase inhibitors (NNRTIs)
   1.26.2. Nucleoside reverse transcriptase inhibitors (NRTIs)
   1.26.3. Protease inhibitors (PIs)
   1.26.4. Fusion inhibitors
   1.26.5. CCR5 antagonists (CCR5s)
   1.26.6. Integrase strand transfer inhibitors (INSTIs)
1.27. Any foregoing method comprising transforming cells containing a retroviral provirus using a vector according to any if Vector 1, et seq. below.

In another embodiment, the invention provides a DNA vector (Vector 1) comprising an expression cassette having a coding sequence encoding a functional MARF1 operatively linked to a promoter, e.g., a heterologous promoter, e.g. wherein the expression cassette can express said coding sequence to provide MARF1 in cells containing a retroviral provirus.
1.1. Vector 1 wherein the DNA vector is a replication-deficient viral vector.
1.2. Vector 1.1 wherein the replication-deficient viral vector is an adenoviral (AV) vector.
1.3. Vector 1.3 wherein the AV vector is not capable of integration into the genome of said cells containing a retroviral provirus.
1.4. Vector 1.1 wherein the replication-deficient viral vector is an adeno-associated viral (AAV) vector, e.g. AAV2, 4, 5, 8, or 9, or a synthetic AAV vector such as AAV-DJ (synthetic serotype made from DNA family shuffling of 8 wild type serotypes of AAV, including AAV2, 4, 5, 8, 9, avian, bovine and goat AAV) or self complementary adeno-associated virus (scAAV).
1.5. Vector 1.4 wherein the AAV vector is not capable of integration into the genome of said cells containing a retroviral provirus.
1.6. Any foregoing Vector wherein the vector is incapable of integrating to the genome of the cells containing a retroviral provirus but will transiently express MARF1 in said cells, e.g., wherein the vector lacks functional elements necessary for integration, e.g., lacks functional rep and/or cap elements.
1.7. Any of Vector 1, 1.1, 1.2 or 1.4 wherein the vector is capable of permanently transforming cells containing a retroviral provirus with a gene constitutively expressing a functional MARF1.
1.8. Any foregoing Vector wherein the MARF1 is a human MARF1.
1.9. Any foregoing Vector wherein the MARF1 has at least 90% amino acid sequence identity, e.g. at least 95% identity, e.g., at least 99% identity, e.g., as measured by a BLAST algorithm, to a MARF1 selected from GenBank Accession: NP_055462.2, NP_001171927.1 and NP_001171928.1.
1.10. Any foregoing Vector wherein the functional MARF1 has at least 90% amino acid sequence identity, e.g. at least 95% identity, e.g., at least 99% identity, e.g., as measured by a BLAST algorithm, to SEQ ID: 1.
1.11. Any foregoing Vector wherein the functional MARF1 comprises one or more, or 5 or more, or all of the following conserved sequences from the sequence of SEQ ID 1: 352-494, 1487-1562, 1004-1074, 1100-1171, 1260-1330, 1176-1247, 1412-1484, 787-875, 510-582, 1336-1406, 876-937, 795-865.
1.12. Any foregoing Vector wherein the functional MARF1 comprises residues 352-1562 of SEQ ID 1.
1.13. Any foregoing Vector wherein the sequence encoding a functional MARF1 is a sequence which encodes a protein which comprises one or more, or 5 or more, or all of the following conserved sequences from the sequence of SEQ ID 1: 352-494, 1487-1562, 1004-1074, 1100-1171, 1260-1330, 1176-1247, 1412-1484, 787-875, 510-582, 1336-1406, 876-937, 795-865; e.g., a protein which comprises residues 352-1562 of SEQ ID 1.
1.14. Any foregoing Vector wherein the sequence encoding a functional MARF1 is a sequence which has at least 90% sequence identity, e.g. at least 95% identity, e.g., at least 99% identity, e.g., as measured by a BLAST algorithm, to the coding sequence (CDS) of a nucleotide sequence selected from GenBank Accession: NM_014647.3, NM_001184998.1, and NM_001184999.1
1.15. Any foregoing Vector wherein the promoter is a heterologous promoter, e.g. a promoter different from a natural MARF1 promoter, e.g. wherein the coding sequence encoding a functional MARF1 and the promoter are derived from different species.
1.16. Any foregoing Vector wherein the promoter is a constitutive promoter.
1.17. Any foregoing Vector wherein the promoter is a viral promoter.
1.18. Any foregoing Vector wherein the promoter is selected from cytomegalovirus (CMV) promoter, chicken (3-actin (CBA) promoter, and CAG promoter (i.e., hybrid of the cytomegalovirus (CMV) early enhancer element and chicken beta-actin promoter).
1.19. Any foregoing Vector further comprising a selectable marker, e.g., an antibiotic resistance gene.

In other embodiments, the invention provides a packaging cell line which expresses a vector as described in Vector 1 et seq. For example, where the vector is a replication-deficient viral vector, the packaging cell line will express in trans the genes required to permit replication of the vector in the cell line. For example, in some embodiments, to introduce high levels of expressed MARF1 in somatic cells, the vector may be an adenoviral (AV) vector, e.g., a serotype 5 adenovirus, wherein the adenovirus is engineered to be nonreplicative and nonpathogenic, e.g., by deletion of all or part of E1 (which is necessary for replication) and E3 (to provide room for insert of the gene of interest). In this case, the vector can be produced in a packaging cell line that is engineered to express the E1 necessary for the virus to replicate. AV vectors are useful because they infect and express in many cell types including nondividing cells and typically will not integrate into the human genome. In other embodiments, AAV vectors may be used, provided the size of the MARF1 expression cassette is small enough to fit in the AAV vector, e.g., less than about 4.7 Kb. In this embodiment, the recombinant AAV will preferably have the replication and capsid genes are provided in trans (in pRep/Cap plasmid), so that only the two ITRs of AAV genome are left and packaged into virion, while the adenovirus genes required are provided either provided by adenovirus or another plasmid, so that risk for the recombinant AAV to replicate in the cells is very low. For example, in one embodiment, the AAV vector is made by co-transfection of AAV production cells with three plasmids: (1) an AAV2 ITR-containing plasmid carrying the MARF1 expression cassette; (2) a plasmid that carries the AAV2 Rep-Cap; and (3) a plasmid that provides the helper genes isolated from adenovirus. In some embodiments, to avoid risk of triggering an immune response to the vector, the vector is administered ex vivo, to blood cells, for example CD4+ cells, which are removed, transfected, and then returned to the body only after the vector titer is substantially reduced. In some embodiments, the vector will comprise a selectable marker, for example an antibiotic resistance gene, wherein the antibiotic is one such as kanamycin which would kill the packaging cell line in the absence of the resistance gene, so that the packaging cell line is under selective pressure to produce the vector. Non-replicating AV and AAV vectors are commercially available from many sources, e.g., from Vector Biolabs (Malvern, Pa.) or Agilent (Santa Clara, Calif.), for example Agilent's AdEasy adenoviral vector system that uses recombination with a phage produced in E. coli to insert the gene of interest.

In some embodiments the invention provides MARF1 or a vector comprising a gene for MARF1, e.g., as described in Vector 1, et seq., as a therapeutic agent, e.g., for treatment of a retroviral infection in a human, e.g., in accordance with Method 1 et seq. The invention further provides the use of a sequence expressing MARF1 in the manufacture of a therapeutic agent, e.g., Vector 1 et seq., e.g., for use in treating a retroviral infection in a human, e.g., in accordance with Method 1 et seq.

Without intending to be bound by theory, it is proposed that the mechanism of MARF1's efficacy to silence a retroviral provirus is similar to the mechanism as seen in MARF1's silencing of retrotransposons in oocytes.

Certain types of transposable elements, for example Class 1 transposable elements or retrotransposons, are first transcribed from DNA to RNA, then the RNA produced is reverse transcribed to DNA. This copied DNA is then inserted at a new position into the genome. The reverse transcription step is catalyzed by a reverse transcriptase, which may be encoded by a coding region in the transposable element. Retroviruses like HIV and some retrotransposons both contain long terminal repeats (LTRs) and encode reverse transcriptase. Indeed, it is thought that retrotransposons may be descended from ancient retroviral infections. The LTRs act to mediate integration of the retroviral DNA via an LTR specific integrase into the host chromosome. A retroviral provirus can thus be understood as a particular type of eukaryotic retrotransposon, which can produce RNA intermediates that, rather than producing DNA which reintegrates into the host genome, will leave the host cell and produce DNA that can integrate into the genome of other cells.

While the exact mechanism is not known, it appears that MARF1 recognizes and stimulates methylation of retrotransposons in oocytes. Once the retrotransposons are methylated, their expression is also suppressed in progeny cells. Thus MARF1 is not normally expressed or needed in somatic cells, because the gene silencing of the retrotransposons is already carried out in the oocyte and persists in the somatic cells. But somatic cells are capable of suppressing expression of genes through methylation or other epigenetic means, so the basic mechanism for suppression normally should be available in somatic cells as in oocytes.

While the regulators for gene expression may vary (primarily MARF1 in oocytes, primarily tumor suppressor factors in somatic cells) the basic machinery for gene suppression (e.g., histone deacetylase and DNA methylase) appears to be ubiquitous, and thus potentially available for use by MARF1 if MARF1 is provided to somatic cells.

Azidothymidine (AZT) is a nucleoside analog reverse-transcriptase inhibitor that was used as an effective antiviral for HIV until a mutant AZT-resistant strain took over. AZT is shown to have a profound effect on fetal oocyte attrition, increasing oocyte numbers in mice, as FOA is largely dependent on favoring oocytes with less LINE-1 retrotransposon activity. This suggests some functional homology between retroviral proviruses and retrotransposons and supports the theory that a mechanism that silences one could be effective to silence the other.

One could think of a cell infected with a retrovirus as facing a problem (a potentially disruptive retrotransposon), which is routinely managed with extremely high efficiency at the oocyte level, but which is only rarely seen at the somatic cell level. The proposal involves bringing a tool from the oocyte (MARF1) to fix an analogous problem in a somatic cell.

In some embodiments, the invention involves administering to HIV positive patients a vector expressing the meiosis arrest female 1 (MARF1) gene, or alternatively, isolating, treating and reintroducing their CD4+ T-cells. The MARF1 should permanently suppress the HIV provirus by selective methylation, so that the provirus will also be suppressed in progeny cells. CD4+ T-cells with HIV provirus suppressed should have a selective advantage over the non-suppressed cells.

Although the expression of the retroviral provirus in a particular cell line will be permanently silenced following exposure to MARF1, there is still some risk of reinfection from cells that are not treated with the MARF1. This risk can be reduced by continued treatment with conventional antiviral therapies, which suppress infection, or it may be desirable to use a vector that will permanently transform cells containing a retroviral provirus with a gene constitutively expressing MARF1, so that such cells will be resistant to subsequent retroviral infection. This approach has the advantage that the transformed cells will have a selective advantage over the nontransformed cells in the presence of the virus, but may also carry additional risks, however, insofar as the integration of the MARF1 transgene may disrupt other genes, and the long-term effect of constitutive MARF1 expression is not known.

The foregoing description of certain preferred embodiment(s) and the following examples are in no way intended to limit the invention, its application, or uses.

### Example 1—Demonstration that MARF1 can Silence Genes in Somatic Cells (CD4+ Cells)

MARF1 should be able to target first a variety of retrotransposons, and then a retrovirus, as a retrovirus is essentially a retrotranspon with envelope protein. Introducing MARF1 into somatic cells should show a decrease in levels of retrotransposon mRNA and when tested with a retrovirus, show a decrease in virus titer.

CD4+ cells (SUP-T1, although Jurkat cells expressing CD4 may be used instead) are transformed using adenovirus/retrotransposon hybrid vectors describe by Kubo et al. to insert a L1RP retrotransposon/GFP indicator transgene. See Kubo, S., & Soifer, H. “779. High-Capacity Adenovirus/Retrotransposon Hybrid Vectors for Efficient and Stable Gene Transfer”. Molecular Therapy, (2004) 9: S295. AV-MARF1 vector is prepared using Adeno-X Expression System 3 (Clontech) and MARF1 cDNA corresponding to the coding sequence (CDS) of GenBank Accession: NM_014647.3.

GFP expression is measured in the transformed cells in combination with (i) AV-MARF1 vector or (ii) AV-blank vector (control). Transfection with AV-MARF1 suppresses GFP expression in the cells.

### Example 2: Efficacy of MARF1-AV Vector in HIV-Infected Cell Line

A CD4+ cell line highly susceptible to HIV infection is created, e.g., generally as described in Krowicka H, Robinson J E, Clark R, Hager S, Broyles S, Pincus S H. Use of Tissue Culture Cell Lines to Evaluate HIV Antiviral Resistance. AIDS Research and Human Retroviruses. 2008; 24(7):957-967. The cells are infected with HIV and then one group is treated with MARF1-AV, the other with a blank AV vector. Levels of HIV Gag polyprotein are monitored at different points after infection. Decrease in polyprotein level will correlate with inhibition of replication. Gag protein levels can be determined using a radioimmunoassay (RIA).

1. A method of treating a retroviral infection in a human in need of such treatment, comprising delivering a functional meiosis arrest female protein 1 (MARF1) to cells containing a retroviral provirus.

2. The method of claim 1, wherein the MARF1 is delivered by means of a DNA vector comprising an expression cassette having a coding sequence encoding a functional MARF1 operatively linked to a promoter, that which will express MARF1 in said cells containing a retroviral provirus.

3. The method of claim 2 wherein the viral vector is a replication-deficient adenoviral (AV) vector or a replication-deficient adeno-associated viral (AAV) vector.

4. The method of claim 1 wherein the retroviral infection is human immunodeficiency virus (HIV).

5. The method of claim 1 wherein the functional meiosis arrest female protein 1 (MARF1) is delivered to the cells containing a retroviral provirus ex vivo, by removing from the patient's body, treating, and returning the cells containing a retroviral provirus.

6. A DNA vector comprising an expression cassette having a coding sequence encoding a functional MARF1 operatively linked to a heterologous promoter.

7. The vector of claim 6 which is a replication-deficient adenoviral (AV) vector or a replication-deficient adeno-associated viral (AAV) vector.

8. A packaging cell line which expresses a vector according to claim 6.

9.-10. (canceled)


SEQUENCE LISTING (1 sequence)

SEQ ID NO: 1 - protein, 1742 aa, Homo sapiens
MMEGNGTENSCSRTRGWLQQDNDAKPWLWKFSNCFSRPEQTLPHSPQTKEYMENKKVAVELKDVPSPLHAGSKLFPAVPLPDIRSLQQPKIQLSSVPKVSCCAHCPNEPSTSPMRFGGGGGGSGGTSSLIHPGALLDSQSTRTITCQVGSGFAFQSASSLQNASARNNLAGIASDFPSMCLESNLSSCKHLPCCGKLHFQSCHGNVHKLHQFPSLQGCTSAGYFPCSDFTSGAPGHLEEHISQSELTPHLCTNSLHLNVVPPVCLKGSLYCEDCLNKPARNSIIDAAKVWPNIPPPNTQPAPLAVPLCNGCGTKGTGKETTLLLATSLGKAASKFGSPEVAVAGQVLENLPPIGVFWDIENCSVPSGRSATAVVQRIREKFFKGHREAEFICVCDISKENKEVIQELNNCQVTVAHINATAKNAADDKLRQSLRRFANTHTAPATVVLVSTDVNFALELSDLRHRHGFHIILVHKNQASEALLHHANELIRFEEFISDLPPRLPLKMPQCHTLLYVYNLPANKDGKSVSNRLRRLSDNCGGKVLSITGCSAILRFINQDSAERAQKRMENEDVFGNRIIVSFTPKNRELCETKSSNAIADKVKSPKKLKNPKLCLIKDASEQSSSAKATPGKGSQANSGSATKNTNVKSLQELCRMESKTGHRNSEHQQGHLRLVVPTHGNSSAAVSTPKNSGVAEPVYKTSQKKENLSARSVTSSPVEKKDKEETVFQVSYPSAFSKLVASRQVSPLLASQSWSSRSMSPNLLNRASPLAFNIANSSSEADCPDPFANGADVQVSNIDYRLSRKELQQLLQEAFARHGKVKSVELSPHTDYQLKAVVQMENLQDAIGAVNSLHRYKIGSKKILVSLATGAASKSLSLLSAETMSVLQDAPACCLPLFKFTDIYEKKFGHKLNVSDLYKLTDTVAIREQGNGRLVCLLPSSQARQSPLGSSQSHDGSSTNCSPIIFEELEYHEPVCRQHCSNKDFSEHEFDPDSYKIPFVILSLKTFAPQVHSLLQTHEGTVPLLSFPDCYIAEFGDLEVVQENQGGVPLEHFITCVPGVNIATAQNGIKVVKWIHNKPPPPNTDPWLLRSKSPVGNPQLIQFSREVIDLLKSQPSCVIPISHFIPSYHHHFAKQCRVSDYGYSKLIELLEAVPHVLQILGMGSKRLLTLTHRAQVKRFTQDLLKLLKSQASKQVIVREFSQAYHWCFSKDWDVTEYGVCELIDIVSEIPDTTICLSQQDNEMVICIPKRERTQDEIERTKQFSKDVVDLLRHQPHFRMPFNKFIPSYHHHFGRQCKLAYYGFTKLLELFEAIPDTLQVLECGEEKILTLTEVERFKALAAQFVKLLRSQKDNCLMMTDLLTEYAKTFGYTFRLQDYDVSSISALTQKLCHVVKVADIESGRQIQLINRKSLRSLTAQLLVLLMSWEGTTHLSVEELKRHYESTHNTPLNPCEYGFMTLTELLKSLPYLVEVFTNDKMEECVKLTSLYLFAKNVRSLLHTYHYQQIFLHEFSMAYTKYVGETLQPKTYGHSSVEELLGAIPQVVWIKGHGHKRIVVLKNDMKSRLSSLSLSPANHENQPSEGERILEVPESHTASELKLGADGSGPSHTEQELLRLTDDSPVDLLCAPVPSCLPSPQLRPDPVILQSADLIQFEERPQEPSEIMILNQEEKMEIPIPGKSKTLTSDSSSSCISAAVPVPPCPSSETSESLLSKDPVESPAKKQPKNRVKLAANFSLAPITKL